(19) United States
(12) Patent Application Publication
(10) Pub. No.: US 2019/0380649 A1
(43) Pub. Date: Dec. 19, 2019

(54) Back and neck orthotic device

(71) Applicants: Gongbo Hu Li, Surrey (CA); Olivia Li, Surrey (CA); Li Nan Ouyang, Surrey (CA)

(72) Inventors: Gongbo Hu Li, Surrey (CA); Olivia Li, Surrey (CA); Li Nan Ouyang, Surrey (CA)

(21) Appl. No.: 16/455,757

(22) Filed: Jun. 28, 2019

(30) Foreign Application Priority Data
Mar. 14, 2019 (CN) 201910192010.2

(51) Int. Cl. A61B 5/00; H01L 41/113; A61B 5/11

(52) U.S. Cl. CPC A61B 5/6812; H01L 41/1132; A61B 5/1123; A61B 5/1116; A61B 5/4566; A61B 2560/0214; A61B 5/746; A61B 5/7455; A61B 5/742; A61B 5/6804; A61B 5/486

(57) ABSTRACT

A novel back and neck orthotic device is disclosed. This back and neck orthotic device includes an elastic matrix layer; an electronic textile layer which electrochemically generates electrical energy when stretched; Additionally, an alert module is mounted to the one side of the elastic matrix layer, detecting when a person bends and alerting them correspondingly. When used, it is only necessary to attach the back and neck orthotic device to person's back and/or neck. When the back or neck is bent, the elastic matrix layer is elastically deformed, resulting in the electronic textile layer is to be deformed as well, allowing the electronic textile to output a voltage. When the output voltage exceeds the preset voltage threshold, the alert module is triggered to remind the user to correct their posture.

### FIELD OF THE INVENTION

The present invention is directed to the field of skin motion sensing devices that monitor unhealthy body especially spinal and head movement. In particular, the present invention helps individual maintain healthy postures no matter if they are sitting or standing by using a self-powered electric matrix layer and biofeedback technology.

### BACKGROUND OF THE INVENTION

The present invention employs an electric matrix layer, for example, triboelectric nanogenerator, that produces energy when bent to detect whether the user is bending their back or neck beyond a certain degree and warns them through a vibrating, audible or visual alarm. The present invention is attached to the user through an adhesive cloth patch on a user's back and neck.

Cell phones and tablets are changing the way we access information and entertainment. The use of these devices influences our posture and body mechanics in unhealthy ways that contribute to neck, upper back, shoulder, and arm pain. Furthermore, poor posture while sitting, standing, walking, or in any static position can lead to more than upper body pain and stiffness—poor posture affects other parts of the spine, such as the middle and lower back.

Kenneth K. Hansraj, M.D., Chief of Spine Surgery at New York Spine Surgery and Rehabilitation Medicine, created a computer model of the cervical spine. This model showed that the strain on one's neck rises as the forward angle of the head increases. 45 degrees forward equals 49 pounds of strain, and 60 degrees forward equals 60 pounds. Making good posture a habit can help prevent neck or back pain from developing, along with other posture and biomechanical problems. Good posture means that one's head is upright, your ears are in line with the shoulders, and the shoulder blades are down and retracted. Good posture is not only good for the health of the spine; it is good for overall health and mood as well. In fact, standing straight elevates testosterone and serotonin levels and while decreasing cortisol levels, hormones that affect your mood.

There is a need, however, for a device that can be attached to a person to retrain their bending habits by consistently and automatically alerting them when they are jeopardizing their upper back and neck health by bending forwards too much. The previously cited art requires external power supply or built-in battery to work and use properly. It also fails to provide a device that can be easy to use, light and self-powering. The alert point can be set based on a certain electric energy threshold. It would therefore be beneficial if such a device exists, as a retraining aid, which detects when a person bends past a certain set point thus triggering the alert.

### OBJECTIVES AND SUMMARY OF THE INVENTION

It is an objective of the present invention to provide a back and neck orthotic device that will alert a user when they bend forward too much, resulting in poor posture.

Additionally, it is an objective of the present invention to provide a back and neck orthotic device that includes an elastic matrix layer, an electronic textile layer that electrochemically generates electrical energy when stretched. Also, an alert module is mounted to the one side of the elastic matrix layer, detecting when the user bends or stretches.

It is a further objective of the present invention to provide a back and neck orthotic device that solves the problem of a complicated and/or heavy structure resulting from an external power supply or built-in battery.

Furthermore, the present invention utilizes a triboelectric nanogenerator and/or other electronic textiles to serve as an energy harvester to generate electric energy to power the alert system.

Lastly, it is a further objective of the present invention to provide a device that can be attached via an adhesive patch to the user's back and neck for increased convenience and comfort.

In accordance with the first aspect of the present invention, a novel back and neck orthotic device is disclosed. The novel back and neck orthotic device includes an elastic matrix layer; an electronic textile layer that electrochemically generates electrical energy when stretched; and an alert module mounted to the one side of the elastic matrix layer, detecting when a person bends or stretches.

In accordance with another aspect of the present invention, a novel back and neck orthotic device includes a triboelectric nanogenerator that harvests energy when the user bends forward; The triboelectric nanogenerator and/or electronic textiles includes: carbon nanotubes and carbon nanotube-based composite conductive materials; graphene; composite conductive materials based on silver powder; or silver sheets or silver nanowires; carbon black-based composite conductive materials; and hydrogel-based conductive materials; and an alert module, operatively connected to the electronic textiles for power supply.

The novel miniature back and neck orthotic device includes a miniature and thin elastic matrix layer or film; an electronic textile layer electrochemically generated electrical energy when stretched; and an alert module mounted to the one side of the elastic matrix layer, for detecting when a person bends or stretches. The miniature whole device is adapted to be directly attached to the skin of the person.

### BRIEF DESCRIPTION OF THE DRAWINGS

The previous summary as well as the following detailed description of a preferred embodiment of the present invention will be better understood when read in reference to the appended drawings, wherein:

FIG. 1 is a side elevation schematic of the novel back and neck orthotic device (10) in accordance with the present invention;

FIGS. 2(a)-2(c) are schematic views showing some alternative shapes of the elastic matrix layer (100) in the novel back and neck orthotic device (10) in accordance with the present invention;

FIG. 3 is an internal side elevation schematic view of an alternative embodiment of the novel back and neck orthotic device (10) in accordance with the present invention

FIGS. 4(a)-4(d) are schematic structural views of an electronic textile layer (200) in the novel back and neck orthotic device (10) in accordance with the present invention.

### DETAILED DESCRIPTION OF THE PREFERRED EMBODIMENT OF THE DEVICE

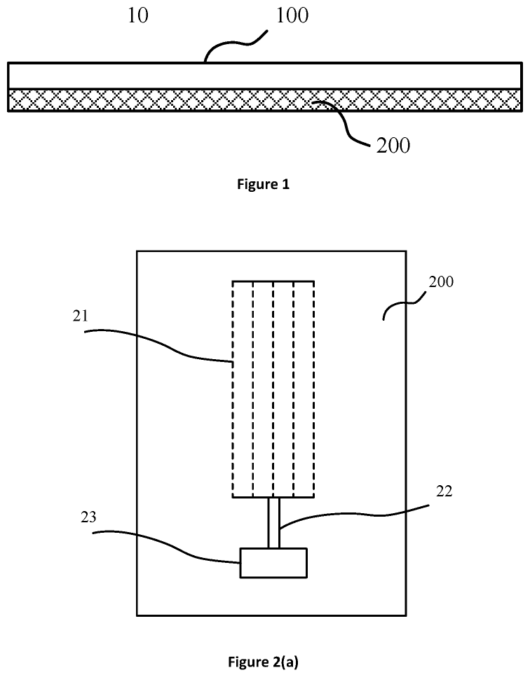

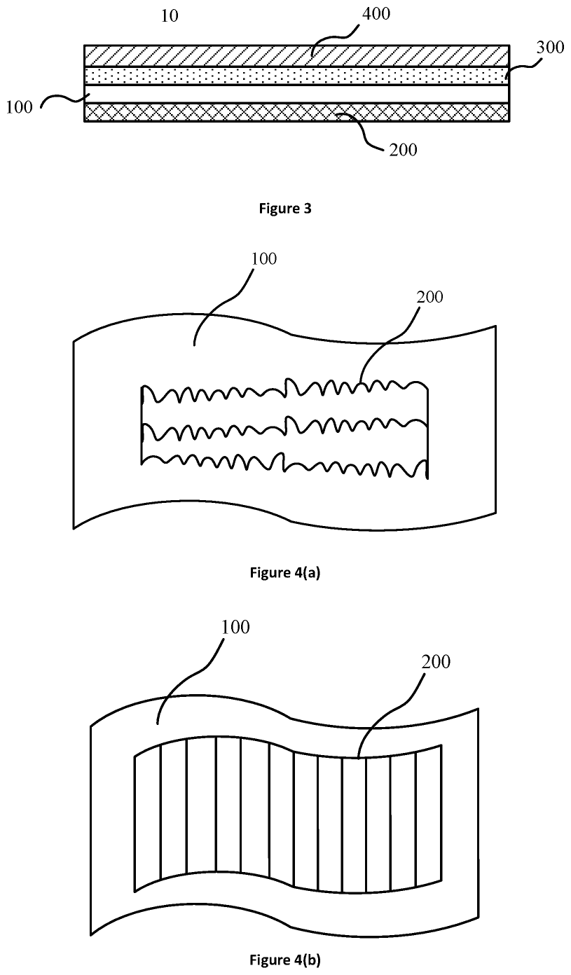

Referring now to the drawings, where the reference numerals refer to the same components across the several views with alternative embodiments of the present invention. In particular, FIGS. 1 and 3, shown the back and neck orthotic device (10). The back and neck orthotic device (10) includes an elastic matrix layer (100), an electronic textile layer (200) that electrochemically generates electrical energy when stretched. In addition, an alert module (23) is mounted to one side of the elastic matrix layer (100), to detect when the user hyperextends their back or maintains bad posture.

U.S. Pat. No. 5,749,838 discloses a posture training device, which includes a frame and a module engaged with the frame. The device can be held snugly against the spine bone of a person by a belt or garment, such that an increase in curvature of the back in either the midsagittal plane or a transverse plane moves the module relative to the frame. When the module has been moved sufficiently to indicate a poor posture position, a tactile signal generator within the module is activated and vibrates, alerting the users that they have assumed a poor posture.

U.S. Patent Application No. 20020170193 discloses a sensing device, which is attached to a living subject that includes sensors for distinguishing lying, sitting, and standing positions. In another embodiment, sensor data is stored in a storage device as a function of time. Multiple points or multiple intervals of the time dependent data are used to direct a feedback mechanism to provide information or instruction in response to the time dependent output indicating too little activity, too much time with a joint not being moved beyond a specified range of motion, too many motions beyond a specified range of motion, or repetitive activity that can cause repetitive stress injury.

U.S. Pat Application No. 20120245491A1 discloses a device for monitoring a posture of a user that includes a belt adapted to encircle the torso of the user and provides an upward force to a spinal region of the user in response to a signal from a controller, and at least one sensor adapted to provide at least one measurement signal to the controller. The device also includes a feedback system activated by the controller to alert the user that a posture of the user is determined to be outside an acceptable range based on the at least one measurement signal. An ergonomic posture assisting system is provided that includes a detachable belt. A detachable belt is provided that includes a locking mechanism adapted to prevent the attachment mechanism from disconnecting in response to a signal. A posture feedback system and a device for monitoring a posture of a user are provided.

U.S. Pat. No. 7,843,351 B1 discloses a back training device that monitors unsafe spinal movement to help individuals to retrain themselves to bend primarily at the knees instead of bending primarily at the waist by using biofeedback technology and Pavlovian response.

U.S. Pat. No. US 9,196,175 B2 discloses a portable system for sensing and training a user to maintain correct posture while seated in a chair.

Canadian Patent No. 2503537 discloses a repositioning device, garment, and posture molding method and training instruction using them. A repositioning device comprises a case provided on a skin side of a garment or being attachable to a human body surface by an adhesive, and thereby adapted to contact said human body surface. A piezoelectric element is provided within the case and adapted to generate vibrations in a range of 3 Hz to 5 Mhz. A coin battery is provided within the case for supplying power to the piezoelectric element, and a controller is provided within the case for controlling generation of said vibrations by the piezoelectric element. The case has a point-like surface of 4 cm² or smaller in contact the skin of the user, and the piezoelectric element is provided at a central part of the point-like surface.

The present invention relates to a self-powered device designed to promote proper posture through biofeedback. The device also may provide a wake-up mechanism for long term computer, cell phone, tablet users, or drowsy drivers. Moreover, the invention provides training on healthy posture of back and neck that reduces the amount of weight and pressure on the user's back and neck. The device also encourages proper posture through posture-related tactile biofeedback to inform the user when their back and neck bent over a preset level. This biofeedback mechanism serves the dual purpose of posture correction and a wake-up mechanism to alert users.

To address structural complications caused by the need for an external power supply or a built-in battery in previous similar orthotic devices, the applicant of this invention has proposed an orthotic device comprising an elastic matrix layer, an electronic textile layer and an alert module. The electronic textile layer is attached to one side of the elastic matrix layer, and the alert module is electrically connected to the electronic textile layer. When used, the orthotic device is attached to the body to be corrected, such as the back or the neck of the user using adhesive. When the back or neck is bent, the elastic matrix layer is elastically deformed, and the electronic textile layer generates electric energy due to such as stretching, twisting or bending which outputs a voltage. When the voltage outputted by the electronic textile layer exceeds a preset threshold (i.e., the electronic textile layer itself can be used as a power source) the alert module is activated and the user is reminded to correct their posture. Therefore, the orthotic device provided by the present invention does not require an external power supply or a built-in power supply, and the structure is very simple. In addition, it is only necessary to attach the orthotic device to the area of the body to be corrected, such as the back or the neck, resulting in convenience and ease of use.

FIG. 1 shows the structure of the orthotic device (10) according to an embodiment of the present invention. For clarity, only parts related to the embodiment of the present invention are shown and the figure is very magnified as the layers are very thin in reality. The embodiment is described in detail as follows:

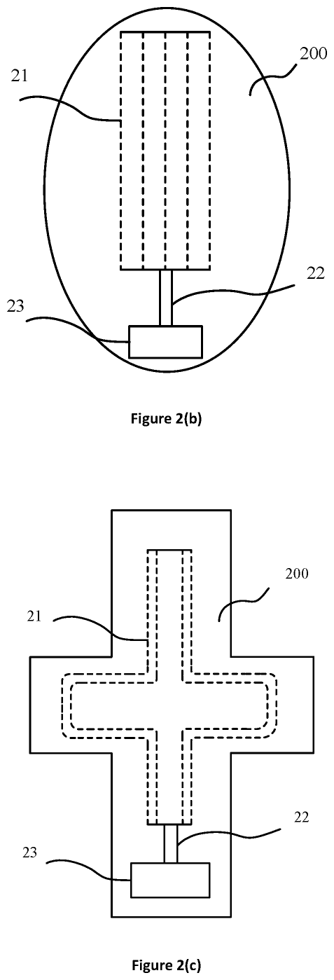

The elastic matrix layer (100), the deformable electronic textile layer (200) and the alert module (23) (not shown in FIG. 1; see FIG. 2(a)-2(c));

The electronic textile layer (200) is attached to one side of the elastic matrix layer (100), and the alert module (23) is electrically connected to the electronic textile layer (200);

The elastic matrix layer (100) is elastically deformed when the neck or back of the user is bent, allowing the electronic textile layer (200) to output a voltage. The alert module (23) uses the output voltage from the electronic textile layer (200) and reminds the user to correct their posture when the output voltage exceeds the preset voltage threshold.

The orthotic device (10) can be an adhesive patch attached to a skin, such as the back or the neck. The orthotic device (10) can also be sewn with a strap. And the orthotic device (10) can be used to securely attach itself to a belt or any wearable clothes. Of course, the orthotic device (10) may be attached to garment, waistband, or a cervical vertebra loop, which will be included but not limited in the embodiments of the present invention.

The Applicants have found that since most similar orthotic devices are not made of elastic material, they result in a poor user experience and discomfort resulting from the tightness. As such, the orthotic device (10) by the embodiment of the present invention adopts an elastic matrix layer (100) capable of deforming elastically and powering itself through the electronic textile layer (200), drastically improving user comfort and the overall user experience.

In addition, the applicant has also found that traditional orthotic devices that also have reminder functions incorporated (such as a bending sensor, alarm, or vibrating motor), all require an external power supply or a built-in battery to work properly. Thus, the traditional orthotic device is complicated in structure. In addition, it is necessary to turn off the power when the correcting device is not used for safety reasons, and often have awkward harness mechanisms that are an inconvenience to the typical wearer.

In view of this, the applicants have attempted to improve the conventionally structurally complicated orthotic device and intends to construct a straightening device with a simple, easy to use design structure. The applicants discovered a yarn that could generate its own power with the name of “Twistron” during the orthotic design research process. The study was published in Science on Aug. 25, 2017, where researchers developed a light, yarn-like substance they call the “Twistron”. It was found that each time the Twistron yarn was stretched and deformed, an electrical potential could be generated, with enough energy to illuminate a small LED. When the crimped Twistron yarn is stretched at a frequency of 30 times per second, a peak electric power of 250 W/kg (yarn weight) can be produced. Moreover, the deformation of the Twistron yarn is positively related to its outputted electrical energy (voltage), that is, the greater the deformation of the Twistron yarn, the greater the electrical energy output; the smaller the deformation, the smaller the outputted electrical energy.

Based on this research, the applicant has applied the material to the electronic textile layer (200) so that it can generate electrical energy when stretched and deformed and deliver it to the rest of the orthotic device (10). That is, the electronic textile layer (200) is attached to one side of the elastic matrix layer (100) and, and electrically connected to the alert module (23). When the position of the back or neck of the user is no longer in a healthy position, the bending of the body causes the elastic matrix layer (100) to be elastically deformed; along with the electronic textile layer (200). This deformation generates an electric energy and when the voltage outputted by the electronic textile layer (200) due to deformation exceeds a preset voltage threshold, the electronic textile layer (200) itself is used as the power source for the alert module (23) to remind the user that their posture should be corrected. When the back or neck is straight, the electronic textile layer (200) does not output electric energy, or the outputted electric energy is insufficient to power the alert module (23) and allow it to operate. Therefore, the electronic textile layer (200) in the orthotic device (10) proposed by the applicant can be used as the power source for driving the alert module (23) when deformed, without an external power supply or built-in battery, making the structure very simple. At the same time, the orthotic device (10) can be attached to the skin wherever needing to be corrected. It is easy to use.

In the embodiment of the present invention, the orthotic device (10) includes an elastic matrix layer (100), an electronic textile layer (200), and an alert module (23) attached to one side of the elastic matrix layer (100). The alert module (23) is connected with the electronic textile layer (200). The orthotic device (10) is attached to the wearer's the back or neck. When wearer's back or neck is bent, the elastic matrix layer (100) becomes elastically deformed, and thus deforming the electronic textile layer (200) along with it. If the deformity causes the electronic textile layer (200) to reach a preset voltage threshold, it outputs a voltage that can be used as a power source to drive the alarm module (23) to operate in order to remind the wearer to correct their posture. In summary, the structure of the present invention is very simple, and no external power supply or built-in power source is required. In addition, the orthotic device only needs to be attached when used making it extremely convenient to use.

In an embodiment of the invention, the elastic matrix layer (100) is made of an elastic fabric.

FIGS. 2(a) to 2(c) respectively show the shape of the elastic matrix layer (100) in the orthotic device (10) provided by the embodiment of the invention. For the convenience of the reader, only the parts related to the embodiment of the invention are shown, as detailed below:

In FIG. 2(a), the elastic matrix layer (100) in the orthotic device (10) is in the form of a strip shape.

In FIG. 2(b), the elastic matrix layer (100) in the orthotic device (10) is in the form of a spun cone shape.

In FIG. 2(c), the elastic matrix layer (100) in the orthotic device (10) is in the form of a cross shape.

In an embodiment of the present invention, as shown in FIGS. 2(a) to 2(c), the electronic textile layer (200) includes one Twistron yarn or multiple Twistron yarns arranged in parallel.

In an embodiment of the present invention, as shown in FIG. 2(a) to FIG. 2(c), the alert module (23) is placed at the edge of the elastic matrix layer (100), that is, as far as possible from the center of the correcting device (10). Thus, the edges provide the least possibility of causing damage to the alert module (23). The alert module (23) may includes a sound alert (such as a speaker, a buzzer, etc.), a vibration alert, or a light alert (for example, a flashing LED).

In an embodiment of the present invention, as shown in FIGS. 2(a) to 2(c), the alert module (23) and the electronic textile layer (200) are electrically connected by the flexible circuit (22). Among them, the flexible circuit (22) (full name: Flex Circuits, AKA Membranous Circuits) is a special circuit composed of electronic components mounted on a flexible substrate.

FIG. 3 shows alternative structure of the orthotic device (10) which are described in detail as follows:

In an embodiment of the present invention, in order to facilitate the use of the orthotic device (10), based on the above-described structure of FIG. 1, the orthotic device 10 further includes (as shown in FIG. 3):

An adhesive layer (300) and a release layer (400) are created. The adhesive layer (300) is are coated on the side of the elastic matrix layer (100) opposite to the electronic textile layer (200), while the release layer (400) is coated on this adhesive layer (300).

Among them, the adhesive layer (300) is a substance with good adhesive properties. The release layer (400) (also known as release film) refers to a film that has characteristics of isolation, protection, and easy peeling.

In the embodiment of the present invention, the release layer (400) is isolated from the protective adhesive layer (300) to provide good adhesive functionality before the orthotic device (10) is used. When the orthotic device (10) is needed, it is only necessary to peel off the release layer (400), and the adhesive layer (300) is adhered to the skin of the body. It is very convenient to use.

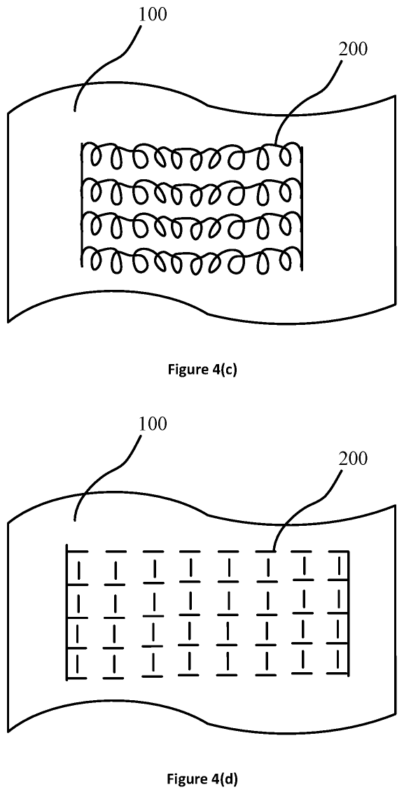

FIGS. 4(a) to 4(d) show the structure of the electronic textile layer (200) in the orthotic device (10). For the convenience of the reader, only parts related to the embodiment of the present invention are shown as follows:

In an embodiment of the present invention, as shown in FIGS. 4(a) to 4(d), the electronic textile layer (200) includes a triboelectric nanogenerator (21). The triboelectric nanogenerator (21) includes: carbon nanotubes and carbon nanotube-based composite conductive materials, graphene, composite conductive materials based on silver powder or silver sheets or silver nanowires, composite conductive materials based on carbon black, and based on a conductive material such as a hydrogel.

In an embodiment of the present invention, as shown in FIG. 4(a), the structure of the electronic textile layer (200) (i.e., the triboelectric nanogenerator (21)) is a pleated structure.

In an embodiment of the present invention, as shown in FIG. 4(b), the structure of the electronic textile layer (200) (i.e., the triboelectric nanogenerator (21)) is a curved structure.

In an embodiment of the present invention, as shown in FIG. 4(c), the structure of the electronic textile layer (200) (i.e., the triboelectric nanogenerator (21)) is a folded structure.

In an embodiment of the present invention, as shown in FIG. 4(d), the structure of the electronic textile layer (200) (i.e., the triboelectric nanogenerator (21)) is a fabric structure.

In summary, the orthotic device (10) includes an elastic matrix layer (100), an electronic textile layer (200), and a alert module (23). The power generating electronic textile layer (200) is attached to one side of the elastic matrix layer (100), and is electrically connected to the alert module (23). The orthotic device (10) is attached to the back or the neck, during use. When the back or neck is bent, the elastic matrix layer (100) is elastically deformed, and the electronic textile layer (200) generates electric energy through the deformation of the elastic matrix layer (100). When this outputted electrical energy exceeds a preset voltage threshold, the alert module (23) is triggered and is used to remind the user to correct their posture. The electronic textile (200) is used to power the alert module, removing the need for batteries or an external power source.

The orthotic device (10) proposed by the present invention has the following beneficial effects:

(1) No external power supply or built-in battery is required;

(2) The structure is very simple;

(3) Only the orthotic device (10) is attached to the body, nothing else. This reduces bulk and weight, allowing it to be portable and convenient;

(4) Constant real-time reminding helps to maintain a good posture;

(5) When the body is bent, the elastic patch can quickly spread the energy to the entire device, improving the accuracy of the unit in determining the bend of the back or neck;

(6) The alert module (23) is disposed on the edge of the elastic matrix layer (100) in the orthotic device (10) which reduces the damage to the orthotic device (10);

(7) Simple structure results in a low cost and makes it easy to store;

(8) Simple working principle that incorporates a safe operation, high sensitivity and high efficiency;

(9) Marketability is high strong and has significant positive economic and social benefits.

While the preferred embodiment of the present invention has been described and illustrated, modifications may be made by one of ordinary skill in the art without departing from the scope and spirit of the invention as defined in the appended claims. For example, instead of using on the back or neck, any part of the body may be employed. Additionally, in a preferred embodiment of the present invention, the alert module may be a preset voltage detector, however any detector known to one of ordinary skill in the art may be utilized. Additionally, the orthotic device would be held by a belt, strap, or other device to the body of the person.

What is claimed is:

1. A back and neck orthotic device, comprising of:
an elastic matrix layer;
an electronic textile layer that electrochemically generates electrical energy when stretched;
an alert module mounted to the one side of the elastic matrix layer that, detects when the user bends their neck or back in an unhealthy position. It is only necessary to attach this orthotic device to the user's back or neck. When the back or neck is bent, the elastic matrix layer is elastically deformed, and the electronic textile layer is deformed within the elastic matrix layer to produce a voltage. When the output voltage is more than the preset voltage threshold, the alert module is triggered to remind the user to fix their posture.

2. The back and neck orthotic device from claim 1, wherein the device further comprising of:
an adhesive layer and a release layer;
The adhesive layer is applied to the side of the elastic matrix layer, opposite to the electronic textile layer, and the release layer is coated on the adhesive layer.

3. The device according of claim 1, wherein the alert module is placed at an edge of the elastic matrix layer.

4. The device of claim 3, wherein the alert module is a vibrating unitor.

5. The device of claim 3, wherein the alert module is a buzzer.

6. The device of claim 3, wherein the alert module is a unit emitting colored light.

7. The device of claim 1, wherein the elastic matrix layer is made of an elastic.

8. The device of claim 7, wherein the shape of the elastic matrix layer is a cross shape.

9. The device of claim 7, wherein the shape of the elastic matrix layer is a spun cone shape.

10. The device of claim 7, wherein the shape of the elastic matrix layer is a strip shape.

11. The device of claim 1, wherein the alert module, and the electronic textile layer are electrically connected by a flexible circuit.

12. The device of claim 11, wherein the electronic textile layer is a material which electrochemically generates electrical energy when stretched.

13. The device of claim 11, wherein the electronic textile layer includes but is not limited to:
Carbon nanotubes and carbon nanotube-based composite conductive materials
graphene
composite conductive materials based on silver powder or silver sheets or silver nanowires
carbon black-based composite conductive materials
hydrogel-based conductive materials.

14. The device of claim 1, wherein the electronic textile layer comprises a stretched wire.

15. The device of claim 1, wherein the electronic textile layer comprises multiple stretched wires arranged in parallel.

16. The device of claim 11, wherein the structure of the electronic textile layer is a pleated pattern.

17. The device of claim 11, wherein the structure of the electronic textile layer is a curved pattern.

18. The device of claim 11, wherein the structure of the electronic textile layer is a folded pattern.

19. The device of claim 11, wherein the structure of the electronic textile layer is a woven pattern.
